(19) United States
(12) Patent Application Publication
(10) Pub. No.: US 2008/0261896 A1
(43) Pub. Date: Oct. 23, 2008

(54) Testosterone generating and metabolizing enhancer

(75) Inventors: Junji Tanaka, Aichi-pref (JP); Hiroshi Shi-Moda, Aichi-pref (JP); Shao-Jie Shan, Aichi-pref (JP); Hiromichi Murai, Aichi-pref (JP)

Correspondence Address: BACON & THOMAS, PLLC, 625 SLATERS LANE, FOURTH FLOOR, ALEXANDRIA, VA, 22314-1176, US

(73) Assignee: Sinphar Pharmaceutical Co., Ltd., I Lan (TW)

(21) Appl. No.: 12/081,147

(22) Filed: Apr. 11, 2008

(30) Foreign Application Priority Data
Apr. 11, 2007 (JP) 2007-103561
May 12, 2007 (JP) 2007-127458

(51) Int. Cl. A61K 31/70; A61P 3/00

(52) U.S. Cl. 514/25

(57) ABSTRACT

A testosterone generating and metabolizing enhancer is proposed in the invention, which increases concentrations of testosterone in both seminal vesicle and bloodstream, and may further promote the gene expression of metabolizing testosterone into 5α-reductase of dihydrotestosterone. The testosterone generating and metabolizing enhancer of the invention is characterized in that an effective component thereof may be phenylethanoid glycosides; said phenylethanoid glycosides may comprise at least one of echinacoside and acteoside, or may comprise both echinacoside and acteoside, more preferably. The testosterone generating and metabolizing enhancer of the invention may be applied in medicines, substances for external uses on skin, and foods and beverages for mammals (including humans); as well as an animal feed for mammals.

### FIELD OF THE INVENTION

The invention relates to a testosterone generating and metabolizing enhancer having novel components, and more particularly to a testosterone generating and metabolizing enhancer that may be applied in medicines, substances for external uses on skin, and foods for mammals including humans.

### DESCRIPTION OF PRIOR ART

Traditionally, it has been known that fresh stems of herbs of the Cistanche family are effective for treating infertility, impotence, and constipation (please refer to Patent Citation 1). Moreover, preparations made from the fresh stems of the perennial herbs may be used to nutritionally supplement the bloodstream and the kidneys. These perennial herbs have been widely cultivated in the northwestern region of China, and are known as “ginseng from the deserts”. Among the cultivated herbs of the Cistanche family, the holoparasitic herb Cistanche tubulosa (Schrenk.) Wight is the most widely cultivated.

Previously, Japanese researchers have systematically studied the chemical compositions and pharmaceutical properties of the herbs of the Cistanche family, and found that phenylethanoid glycosides are the main active components of these perennial herbs (for example, refer to Non-patent Citation 1-3). The active components may serve as effective anti-oxidants, metabolizing enhancers, memory enhancers, and sexual drive enhancers. Many researchers have further studied the medicinal properties of phenylethanoid glycosides.
Patent Citation 1: Japanese Publication Number 2004-250449
Non-patent Citation 1: Sato T, et al. Yakugaku Zasshi, 1985, 105(12): 1131
Non-patent Citation 2: Jimenez C., et al. Nat. Prod. Rep., 1994, 11 (6): 591
Non-patent Citation 3: Cometa F., et al. Fitoterapia, 1993, 64 (3): 195
Non-patent Citation 4: Deng Min et al. Acta PharmacoeSin, 2004, 25(10): 1276-128

### SUMMARY OF THE INVENTION

### Problems to be Solved

Basing on the aforesaid studies, the inventor of the invention had carried out relevant tests, in which rats were fed phenylethanoid glycosides and extracts from herbs of the Cistanche family comprising phenylethanoid glycosides, and the resulted showed that testosterone concentration in the seminal vesicles of the rats was increased. In addition, it was found that after feeding mice the aforesaid substances, testosterone concentration in the bloodstream of the mice was also increased, which indicated that phenylethanoid glycosides and extracts from herbs of the Cistanche family comprising phenylethanoid glycosides may promote the gene expression of turning cholesterol into the enzymes of testosterone, as well as promoting the gene expression of synthesizing 5α-reductase (which allows testosterone to be metabolized into dihydrotestosterone); the results formed the basis for the present invention. In other words, a major objective of the invention is to propose a testosterone generating enhancer, which may enhance the gene expression of testosterone generation, and consequently increase testosterone concentration in the seminal vesicle and the bloodstream. Additionally, another objective of the invention is to propose a testosterone metabolizing enhancer, which may enhance the gene expression of 5α-reductase (which allows testosterone to be metabolized into dihydrotestosterone), thereby promoting the metabolism of testosterone into dihydrotestosterone.

### Means for Solving the Problems

In order to solve the aforesaid problems, the invention proposes:

(1) A testosterone generating enhancer comprising phenylethanoid glycosides as effective components.
(2) The testosterone generating enhancer of (1), characterized in that said phenylethanoid glycosides of the testosterone generating enhancer comprise at least one of echinacoside and acteoside.
(3) The testosterone generating enhancer of (2), characterized in that said phenylethanoid glycosides of the testosterone generating enhancer comprise both echinacoside and acteoside.
(4) A testosterone generating enhancer comprising extracts from plants of the Cistanche family as effective components.
(5) The testosterone generating enhancer of (4), characterized in that said extracts comprise phenylethanoid glycosides as effective components.
(6) The testosterone generating enhancer of (5), characterized in that said phenylethanoid glycosides of the extracts comprise at least one of echinacoside and acteoside.
(7) The testosterone generating enhancer of (6), characterized in that said phenylethanoid glycosides of the extracts comprise both echinacoside and acteoside.
(8) A testosterone metabolizing enhancer comprising phenylethanoid glycosides as effective components.
(9) The testosterone metabolizing enhancer of (8), characterized in that said phenylethanoid glycosides of the testosterone metabolizing enhancer comprise at least one of echinacoside and acteoside.
(10) The testosterone metabolizing enhancer of (9), characterized in that said phenylethanoid glycosides of the testosterone metabolizing enhancer comprise both echinacoside and acteoside.
(11) A testosterone metabolizing enhancer comprising extracts from plants of the Cistanche family as effective components.
(12) The testosterone metabolizing enhancer of (11), characterized in that said extracts comprise phenylethanoid glycosides as effective components.
(13) The testosterone metabolizing enhancer of (12), characterized in that said phenylethanoid glycosides of the extracts comprise at least one of echinacoside and acteoside.
(14) The testosterone metabolizing enhancer of (13), characterized in that said phenylethanoid glycosides of the extracts comprise both echinacoside and acteoside.
(15) A medicine applicable to mammals including humans, and the medicine may comprise any of the testosterone generating enhancers described in (1) to (7), or any of the testosterone metabolizing enhancers described in (8) to (14) as effective components.
(16) A substance for external uses on skin applicable to mammals including humans, and the substance for external uses on skin may comprise any of the testosterone generating enhancers described in (1) to (7), or any of the testosterone metabolizing enhancers described in (8) to (14) as effective components.
(17) A food or beverage, and the food or beverage may comprise any of the testosterone generating enhancers described in (1) to (7), or any of the testosterone metabolizing enhancers described in (8) to (14) as effective components.
(18) An animal feed for mammals, which may comprise any of the testosterone generating enhancers described in (1) to (7), or any of the testosterone metabolizing enhancers described in (8) to (14) as effective components.

### DESCRIPTION OF THE ACCOMPANYING DRAWINGS

FIG. 1 is a graph that shows effects Examples 1-3 of the invention have on testosterone generation of the mouse Leydig tumor cell line R2C (mean ±S.D.).

FIG. 2 is a graph that shows the result of P450 SCC gene expression in mice livers tested using RT-PCR. The result of P450 SCC gene expression in FIG. 2 was obtained by repeatedly feeding the mice extracts of cistantubuloside, and indicated as mean ±S.D. (n=3-7).

FIG. 3 is a graph that shows the result of 17α-hydroxylase gene expression in mice livers tested using RT-PCR. The result of 17α-hydroxylase gene expression in FIG. 3 was obtained by repeatedly feeding the mice extracts of cistantubuloside (400 mg/kg), and indicated as mean ±S.D. (n=3-7).

FIG. 4 is a graph that shows the result of 17p-hydroxysteroid dehydrogenase gene expression in mice livers tested using RT-PCR. The result of 17p-hydroxysteroid dehydrogenase gene expression in FIG. 4 was obtained by repeatedly feeding the mice extracts of cistantubuloside (400 mg/kg), and indicated as mean ±S.D. (n=3-7).

FIG. 5 is a graph that shows the result of 5α-reductase gene expression in mice livers tested using RT-PCR. The result of 5α-reductase gene expression in FIG. 5 was obtained by repeatedly feeding the mice extracts of cistantubuloside (400 mg/kg), and indicated as mean ±S.D. (n=3-7).

FIG. 6 is a flow chart that shows the relationship between the enzymes indicated in FIGS. 2 to 5, which also illustrates the pathways of testosterone synthesis from cholesterol and dihydrotestosterone synthesis from metabolizing testosterone in the liver. The abbreviation “HSD” stands for hydroxysteroid dehydrogenase.

### PREFERRED EMBODIMENTS

The invention will be further elucidated hereafter. The testosterone generating and metabolizing enhancer of the invention is characterized in that phenylethanoid glycosides are included as the effective components. In addition, “the testosterone generating and metabolizing enhancer” of the invention refers to both “the testosterone generating enhancer” and “the testosterone metabolizing enhancer”.

The aforesaid phenylethanoid glycosides refer to the following compound having the formula (1).

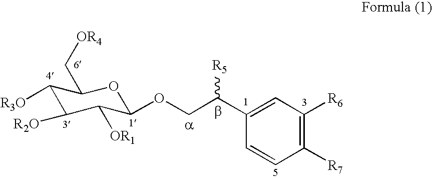

The phenylethanoid glycosides are not limited to the aforesaid scope, and may also include components like echinacoside, acteoside, 2′-acetylacteoside, campneoside I, campneoside II, cistantubuloside A, cistantubuloside B1, cistantubuloside B2, cistantubuloside C1, cistantubuloside C2, crenatoside, decaffeoylacteoside, isoacteoside, rhodioloside, syringalide A 3′-α-L-rhamnopyranoside, and tubuloside A. In addition, the invention may use only one of the aforesaid compounds, or use two or more of the aforesaid compounds.

The phenylethanoid glycosides of the testosterone generating and metabolizing enhancer preferably comprise at least one of echinacoside and acteoside, and more preferably comprise both echinacoside and acteoside, which further enhances the generation and metabolism of testosterone.

The preparation of phenylethanoid glycosides is not limited in any way, and may be obtained by synthesis or extracted from plants. Moreover, if phenylethanoid glycosides are extracted from plants, the plants used as the raw materials for such extraction are not limited in any way, either. For example, the plants used for extraction may include Rehmannia glutinosa (Gaertn.)Libosch. Ex Fisch. Et Mey, plants of genus Stachys (family Lamiaceae), fruits of Piper Linn. (family Piperaceae), and herbs of the Cistanche family. Furthermore, the phenylethanoid glycosides may be extracted from only one of the aforesaid plants, or from two or more of the aforesaid plants. Among the aforesaid plants, the herbs of the Cistanche family are most preferably used for the extraction; this is because the phenylethanoid glycosides from the herbs of the Cistanche family not only include echinacoside and acteoside, but also include 2′-acetylacteoside, campneoside I, campneoside II, cistantubuloside A, cistantubuloside B1, cistantubuloside B2, cistantubuloside C1, cistantubuloside C2, decaffeoylacteoside, isoacteoside, rhodioloside, syringalide A 3′-α-L-rhamnopyranoside, and tubuloside A, and may further enhance the generation and metabolism of testosterone. The plants of the Cistanche family used as the raw materials for extraction are not limited in any way. For example, the plants used for extraction may include Cistanche tubulosa, Cistanche salsa, Aeginetia indica, and Boschniakia rossica, but are not limited thereto. The invention may use only one of the aforesaid plants, or use two or more of the aforesaid plants. However, the holoparasitic plant Cistanche tubulosa is the most preferably used.

The relationship between a composition of the aforesaid phenylethanoid glycosides and the above-mentioned formula (1) is shown below, while the chemical structure of crenatoside is as indicated in the formula (2). Said preparations basically comprise very small or minute amount of components other than the echinacoside and acteoside described above.

TABLE 1
Main components of the testosterone generating and metabolizing
enhancer

| | |
|---|---|
| | Formula (2) |
| 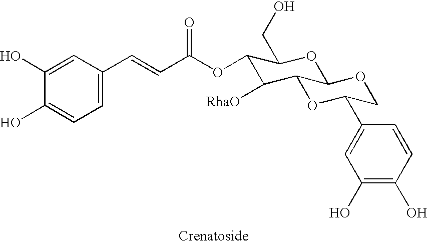 | |

| Components | R₁ | R₂ | R₃ | R₄ | R₅ | R₆ | R₇ |
|---|---|---|---|---|---|---|---|
| 2′-Acetylacteoside | Ac | Rha | Cf | H | H | OH | OH |
| Acteoside | H | Rha | Cf | H | H | OH | OH |
| Campneoside I | H | Rha | Cf | H | OMe(S/R) | OH | OH |
| Campneoside II | H | Rha | Cf | H | OH(S/R) | OH | OH |
| Cistantubuloside A | H | Rha | Cf | Glc | H | H | OH |
| *Cistantubulosides B₁/B₂ | H | Rha | Cm/c-Cm | Glc | H | OH | OH |
| *Cistantubulosides C₁/C₂ | H | Rha | Cf | Glc | OH(S/R) | OH | OH |
| Decaffeoylacteoside | H | Rha | H | H | H | OH | OH |
| Echinacoside | H | Rha | Cf | Glc | H | OH | OH |
| Isoacteoside | H | Rha | H | Cf | H | OH | OH |
| Rhodioloside (Salidroside) | H | H | H | H | H | H | OH |
| Syringalide A 3′-α-L-rham | H | Rha | Cf | H | H | H | OH |
| nopyranoside | | | | | | | |
| Tubuloside A | Ac | Rha | Cf | Glc | H | OH | OH |

| | |
|---|---|
| Crenatoside | Refer to the formula above. |

The compounds shown in the above-mentioned Table 1 may be identified by using high performance liquid chromatography (HPLC). The conditions for using HPLC are shown below. The stationary phase of HPLC was based on stearyl-silance bonded silica gel, while the mobile phase was based on acetonitrile-0.05M phosphoric acid solution (gradient elution was 4:96 15:85), and the flow rate was 1 ml/min; the detection wavelength was set at 330 nm.

The other testosterone generating and metabolizing enhancers of the invention are characterized in that extracts from the plants of the Cistanche family are included as the effective components.

The plants of the Cistanche family used as the raw materials were not limited to any particular plants. For example, the plants may include Cistanche tubulosa, Cistanche salsa, Aeginetia indica, and Boschniakia rossica, but are not limited thereto. The invention may use only one of the aforesaid plants, or use two or more of the aforesaid plants. However, the holoparasitic plant Cistanche tubulosa is the most preferably used.

Furthermore, said “extracts” are preferably comprised of phenylethanoid glycosides. It should be noted that said phenylethanoid glycosides are not limited to any particular compounds, and may include echinacoside, acteoside, 2′-acetylacteoside, campneoside I, campneoside II, cistantubuloside A, cistantubuloside B1, cistantubuloside B2, cistantubuloside C1, cistantubuloside C2, crenatoside, decaffeoylacteoside, isoacteoside, rhodioloside, syringalide A 3′-α-L-rhamnopyranoside, and tubuloside A. In addition, the aforesaid components may be comprised of only one of the aforesaid compounds, or of two or more of the aforesaid compounds. The components preferably comprise at least one of echinacoside and acteoside, and more preferably comprise both echinacoside and acteoside.

The concentration of echinacoside is not specifically limited; assuming that a total weight of the extracts of a plant of the Cistanche family (such as Cistanche tubulosa) was 100 wt %, echinacoside may make up 10-70 wt %, and preferably makes up 15-50 wt %, or more preferably makes up 20-40 wt %, with 23-38 wt % as the most preferable percentage. Moreover, the concentration of acteoside is not specifically limited; assuming that a total weight of the extracts of a plant of the Cistanche family was 100 wt %, acteoside may make up 1-40 wt %, and preferably makes up 5-30 wt %, or more preferably makes up 7-20 wt %, with 8-10 wt % as the most preferable percentage.

The extraction from the plants of the Cistanche family is not limited to any particular parts of the plants, and may be from leaves, roots, and stems; as long as a desired effect is achieved. The extraction is preferably obtained from stems of the plants of the Cistanche family, and especially from fresh stems.

Methods for extracting from the plants of the Cistanche family are not specifically limited, and may be carried out by using solvent extraction and supercritical fluid extraction.

When using the method of solvent extraction, the solvents used are not limited to any particular solvents, but polar solvents are preferably used. The polar solvents are not particularly limited to any solvents, and may be water, methanol, ethanol, isopropanol, acetone, 1,3-butanediol, ethylene glycol, propylene glycol, glycerin, acetic acid, ethyl acetate, and ether. The extraction may use only one of the aforesaid solvents, or use two or more of the aforesaid solvents. Preferably, the extraction is followed by dilution, concentration, drying, and purification; this is because these treatments facilitate the production of phenylethanoid glycosides extracts at high concentration. The purification step may be carried out by using active carbon processing, resin adsorption processing, ion-exchange resins, and liquidi-liquid reversed flow partitioning.

An embodiment for preparing the aforesaid extracts is described in details below, but the preparation of the extracts is not limited to the following embodiment.

The preparation of the extracts comprised of an extraction step and a purification step. In the first step of the preparation, stems (more preferably to be fresh stems) from a plant of the Cistanche family (such as the holoparasitic plant Cistanche tubulosa) were sliced into thin slices, or pulverized into minute particles or powder. The resulted slices, minute particles or powder were immersed in solvents like water, and low-grade fatty alcohols like methanol or ethanol, or a mixed solution thereof. The extraction step was carried out under room temperature. Subsequently, the mixed solution was filtered, and extracts were obtained by concentrating the filtered solution under reduced pressure or in vacuum. In the second step of the preparation, the extracts were heated in water, and then purified by allowing the extracts to pass through adsorption columns filled with Type D-101 or Type AB-8 large-hole adsorption resins. This was followed by eluting the adsorption columns with water, methanol, ethanol, mixed solutions of water and methanol or water and ethanol as the eluting solvents. The eluting process might be done by using the aforesaid eluting solvents of a predetermined concentration, or the aforesaid eluting solvents might be used according to a concentration gradient. The eluting liquids were collected and concentrated, and then dried by using common drying methods. The aforesaid extracts were obtained after the eluting liquids were dried. The resulted extracts were comprised of phenylethanoid glycosides.

The testosterone generating and metabolizing enhancer of the invention may be used as a raw material of a variety of foods and beverages. For example, the foods and beverages may include edible oils (salad oils), snacks (chewing gums, candies, caramel, chocolates, biscuits, bite-sized cookies, jellies, chewing sweets, and desserts), noodles (soba noodles, oolong noodles, and ramen noodles), dairy foods (milk, ice cream, and yoghurt), seasoning foods (miso sauces and bean pastes), soups, drinks (juices, coffee, red tea, tea, carbonated drinks, and sport drinks), which are the general foods, and may further include health foods (tablets and capsules) and nutritional supplements (nutritional drinks). The aforesaid foods and beverages may be adequately supplemented with the testosterone generating and metabolizing enhancer of the invention.

The foods and beverages may have various ingredients added thereto. For instance, the ingredients may include edible food additives like glucose, fructose, sucrose, maltose, sorbitol, stevioside, corn syrup, lactose, citric acid, tartaric acid, malic acid, succinic acid, lactic acid, L-ascorbic acid, dl-α-vitamin E, sodium erythorbate, glycerin, propylene glycol, glycerin fatty acid esters, polyglycerol ester of fatty acids, sucrose fatty acid esters, sorbitol fatty acid esters, propylene glycol fatty acid esters, Arabia gum, carrageenan, casein protein, gelatin, pectin, agar, vitamin B group, nicotinamide, calcium pantothenate, amino acids, calcium salts, colorants, aroma chemicals, and preservatives. In addition, the testosterone generating and metabolizing enhancer of the invention may have other anti-oxidative substances or health foods added thereto to serve as health supplements. The health supplements may include anti-oxidative substances, ascorbic acid of reducing property (vitamin C), vitamin E, glutathione of reducing property, tocotrienol, derivatives of vitamin A, lycopene, β-zeaxanthin, astaxanthin, zeaxanthin, fucoxanthin, uric acid, ubiquinone, coenzyme Q10, folic acid, extracts of garlic, allicin, sesamolin, cellulose, catechin, isoflavone, chalcone, tannic acid, flavone, coumarin, isocoumarin, bilberry extracts, arbutin, tannins, anthocyanin, apple polyphenols, grape seed extracts, ellagic acid, kojic acid, Seabuckthorn extracts, vitamin A, vitamin B1, vitamin B2, vitamin B6, vitamin B12, vitamin C, vitamin D, vitamin E, vitamin P, choline, nicotinamide, pantothenic acid, calcium folinate, EPA, oligosaccharides, food fibers, squalene, soy lecithin, taurine, Dunaliella, proteins, octacosanol, DHA, egg yolk lecithin, linoleic acid, lactoferrin, magnesium, zinc, chromium, selenium, potassium, ferrihemoglobin, extracts of oyster flesh, deacetylated chitin, chito-oligosacchrides, collagen, chondoritin, tulips, licorice, Fructus lycii, cinnamon, crab apples, fresh ginger, Ganoderma lucidum, extracts of shellfish, Trionychidae species, licorice, Fructus lycii, cinnamon, crab apples, fresh ginger, Ganoderma lucidum, Plantago asiatica, Chrysanthemum, Camomiles, dandelion, Hibiscus, honey, spinach, royal jelly, limes, lavender, fruits of wild rose, Rosemary, Sage, Bifidobacterium, faecalis, spore-bearing lactic acid bacteria, wheat germ oil, castor oil, perilla oil, soy oil, middle-chain fatty acids, Agaricus, extracts of Ginkgo leaves, tulips, chondoritin, extracts of germ, Ganoderma lucidum, onions, DHA, EPA, DPA, sweet tea, Cordycrps sinensis (Berk.) Sacc., garlic, bee larvae, papaya, Pu'er tea, propolis, Acer nikoense Maxim, Hericium erinaceum, royal jelly, extracts of Saw palms, hyaluronic acid, collagen, GABA, seal oil, shark cartilage, glucosamine, lecithin, phosphatidylserine, Panax notoginseng (Burk.), mulberry leaves, extracts of soy beans, plants of the Echinacea genus, Siberian ginseng, extracts of barley, olive leaves, olive fruits, Gymnema, Lagerstroemia speciosa (L.)Pers., Salacia, Garcinia, deacetylated chitin, Hypericum perforatum, Fructus Jujubae, ginseng, Passiflora edulis, cauliflower, placenta, Coix lacryma-jobi, grape seeds, peanut peels, bilberry, Black Cohosh, Milk Thistle, bay tree, Sage, Rosemary, Apocynum venetum L., black vinegar, Bitter melon, Maca, safflower, flax, oolong tea, Oxytropis grandiflora, caffeine, capsaicin, xylooligosaccharide, glucosamine, soba, plants of the Citrus genus, food fibers, proteins, dried plum, spirulina, fresh leaves of barley, nucleic acids, yeasts, mushrooms, plum flesh, amino acids, extracts of sharks, noni, oyster flesh, Trionychidae species, Agaricus campestris, Plantago asiatica, cherries, pineapples, bananas, peaches, apricots, melons, strawberries, berries, oranges, fucoidan, Phellinus linteus, cranberries, chondroitin sulfate, zinc, iron, ceramide, silk peptide, glycine, nicotinic acid, Chaste Tree, ceramide, L-cysteine, L-camitine, leaves of red grape, maize, Equisetum debile Roxb, biotin, Gotu Kola, Lonicera caerulea L., pycnogenol, Petasites japonicus, Rheum palmatum L., plants of the Syring a species, Rosemary, catechins, Pu'er tea, citric acid, beer yeast, Melilot, black soybean, fresh ginger, Rhizoma Curcumae Aeruginosae, Bacillus natto, red yeast rice, tocotrienol, lactoferrin, cinnamon, F. tataricum, cocoa powder, Citrus grandis seed extracts, perilla seed extracts, Litchi seed extracts, evening primrose extracts, brown rice extracts, α-lipoic acid, GABA, extracts of raw coffee beans, extracts of Petasites japonicus, kiwi seed extracts, Unshu orange extracts, fresh ginger extracts, and astaxanthin.

For the preparation process, the testosterone generating and metabolizing enhancer (if it is the extract, the extract should be process with cyclodextrin) of the invention may be directly made into powder, granules, tablets, or solutions by using spray drying or freeze drying, so that the products may be readily included into foods (such as instant foods). Moreover, the testosterone generating and metabolizing enhancer or extracts of the invention may also be mixed with adhesive agents like Arabia gum, so as to make the products into powder or granules, and add into solid foods. For the extracts, it may be directly added into beverages, or dissolved in solutions like water, ethanol, glycerin, or a mixture thereof, before adding the extracts into beverages.

The testosterone generating and metabolizing enhancer of the invention may also be used as raw materials for medicines (which includes oral medicines and medicines for external applications). The aforesaid medicines can be manufactured by appropriately adding the testosterone generating and metabolizing enhancer of the invention into raw materials for making the medicines. The aforesaid medicines may be applied to humans, and also other mammals other than the humans. The raw materials that may be used in combination with the testosterone generating and metabolizing enhancer of the invention for making the medicines include: emulsifying agents (glucose, lactose, sugar, sodium chloride, starch, calcium carbonate, kaolin, crystal cellulose, cocoa butter, plant hardening oil, kaolin, and talc powder), adhesive agents (distilled water, normal saline solution, ethanol solution, sugar solution, glucose solution, starch solution, gelatin solution, carboxymethyl cellulose, potassium phosphate, and polyvinylpyrrolidone), disintegrating agents (sodium alginate, agar, sodium bicarbonate, calcium carbonate, dodecyl sodium sulfate, glycerol stearate, starch, lactose, Arabia gum powder, gelatin, and ethanol), anti-disintegrating agents (sugar, stearate, cocoa butter, and hydrogenated oils), absorption promoters (quaternary ammonium hydroxide and dodecyl sodium sulfate), adsorption agents (glycerin, starch, lactose, kaolin, clay, and silicate), and lubricants (fine talc powder, stearate, and polyethylene glycol).

The testosterone generating and metabolizing enhancer of the invention may be orally administered as slices, tablets, soft capsules, hard capsules, granules, powder, and particles. In addition, the testosterone generating and metabolizing enhancer may also be administered as solution-based doses, and the solution-based doses may be administered orally or non-orally. For the non-oral administration, the testosterone generating and metabolizing enhancer may be dissolved in appropriate solvents like ethanol or water, and used as cataplasms, washing solutions, pastes, tinctures, and ointments. The aqueous solutions of the testosterone generating and metabolizing enhancer may be used directly, or have dissipating agents, suspending agents, or stabilizing agents added thereto, before being administered as cataplasms, washing solutions, pastes, tinctures, and ointments.

The administered dosage may vary according to administrative methods, patients' conditions, and patients' age. But an adult is generally administered a daily effective dosage of 5-400 mg, and a child is administered a daily effective dosage of 0.5-200 mg.

When the testosterone generating and metabolizing enhancer is applied for medicinal uses, a ratio thereof may be altered according to the types of administration. Generally, when the orally administered dosage is absorbed through membranes, a ratio for adding the testosterone generating and metabolizing enhancer may be approximately 0.01-10 wt %; but when it is not administered orally, a ratio for adding the testosterone generating and metabolizing enhancer may be approximately 0.01-20 wt %. Because the administrative dosage varies according to different conditions; in some cases, the administrative dosage may be sufficient even if it is smaller than the aforesaid ranges of administrative dosage, or the administrative dosage may have to be more than the aforesaid ranges of administrative dosage. In addition to the testosterone generating and metabolizing enhancer, the medical composition may also comprise other known compounds commonly used for medical purposes, as well as compounds that are commonly used in the orally administered drugs. The compounds may include lactose, starch, hydroxypropyl cellulose, kaolin, talc powder, and calcium carbonate.

The testosterone generating and metabolizing enhancer may also be included into animal feeds for mammals. The testosterone generating and metabolizing enhancer may be included in said animal feeds the same way it is included in the aforesaid foods and beverages. The animal feeds are not limited to uses for any particular animals. For instance, the animal feeds may be formulated to feed farm animals like cattle and pigs, and companion animals like dogs, cats, and hamsters. The animal feeds may include flour and meat as ingredients. Said flour may be comprised of wheat powder, rice powder, rye powder, oat powder, barley powder, grain powder, corn powder, and soy powder; said flour may also be comprised of two or more of the aforesaid powder. The use of said flour may provide the necessary carbohydrates for the companion animals. Among the aforesaid powder, the wheat powder is preferably used. The wheat powder may be used alone or used with high-grade flour, middle-grade flour, and low-grade flour; the wheat powder may also be used with any other types of flour. The elasticity of the heat-processed animal feeds may be adjusted by combining the wheat powder with wheat gran and soy proteins. After the heat treatment, the lattice-like structures in wheat bran will become enlarged, which helps improve its taste.

The meat used in the invention is not specifically limited, and may use chicken, pork, beef, mutton, goat meat, rabbit meat, turkey, and horse meat; chicken is preferably used. The meat may be obtained by using the conventional methods for slaughtering and cutting commonly available farm animals. The deterioration of quality in animal feeds having middle level or low level of water is due to the oxidation of fat, and the solution is to use lean meat that has less or no amount of fat. The composition of meat in the animal feeds not only provides animal proteins of decent quality for the animals being fed, but also improves the behavior of the companion animals.

Though the animal feeds for the companion animals may be fabricated by using a variety of methods, the preferable methods include the following: The animal feeds may be fabricated with a mixture (referred to as “raw dough” hereafter) of raw materials that include flour (with wheat powder as the preferable choice) and meat, and heated after being shaped. The raw dough has flour and meat in a composition thereof, and a ratio between the flour and meat is not specifically limited. Generally, the composition has approximately 5-60% of flour and 5-80% of meat, while the preferably percentage of flour is 10-50%, and the preferable percentage of meat is 20-50%; the raw dough also has water of a desired amount in the composition. When an Aw adjusting agent is used, the Aw adjusting agent takes up 5-30% in the composition, and is preferably between 10-20%. The percentage of water may be adjusted according to the levels that the raw dough is mixed, stirred, and shaped, and also according to the percentage of flour, meat, and the Aw adjusting agent. The methods for fabricating the raw dough is not specifically limited, and the meat is preferably made into fillings by using cutting machines or grinders. In order to sufficiently fill the meat used for making the fillings with bubbles, the meat is preferably sliced into thin slices. The meat used for making fillings are added with flour, water, and the Aw adjusting agent according to actual needs, and then sufficiently stirred to fill in bubbles, so as to make raw dough that contains bubbles. Foaming agents may also be added when fabricating the raw dough, and is preferably used when the aforesaid grain powder except the wheat powder is used. The addition of the foaming agents allows tiny bubbles to be evenly filled into the raw dough. The foaming agents may be all types of foaming agents, but the foaming agents based on soy proteins and/or soy proteins that have been digested with enzymes are preferably used, which is for stabilizing the bubbles.

After shaping and heating the raw dough described above, the animal feeds for the aforesaid companion animals are obtained consequently. When shaping the raw dough, the aforesaid animal feeds may be shaped into appropriate shapes according to factors like facilitating the eating process of the companion animals, and facilitating the feeding process of the owners of such animals. For example, the animal feeds may be shaped into slices, rods, round slices, rings, and heart-shape. Moreover, the raw dough that have been fabricated by using identical methods may be colored with colorants of different tones of color; vegetable and fruits may also be added thereto in order to fabricate raw dough that possess a variety of appearances, and the various raw dough can be combined to form multiple layers or concentric circles as the final shapes. Methods for heating the shaped raw dough are not specifically limited, and may be carried out by using ovens or microwave ovens. The heating methods are commonly known and can be done by the commonly known procedures. After heating, the animal feeds usually comprise approximately 20-40% of water therein. The raw dough also becomes blown up because the heating treatment has caused water therein to evaporate and the bubbles to expand; additionally, because water was evaporated quickly, the Aw of the raw dough was lowered and thus making it more preservable. Furthermore, if wheat powder was used as the flour, the heat treatment would fix the lattice-like structures in wheat bran of the wheat powder, which helps improve its taste. By using an oven for heating the raw dough, a resulted animal feed will appear in a unique color tone (light brown) or be imparted with a unique smell. When a microwave oven is used for heating, because the raw dough is heated from within, the raw dough will become evenly blown, and a resulted animal feed will have evenly distributed bubbles therein. When carrying out the heating treatment, the Aw of the resulted animal feed is preferably between 0.6-0.9. By keeping the Aw within this range, the animal feed may be made more preservable.

The animal feeds fabricated by using the aforesaid methods are made bread-like, and because the animal feeds have soft taste, soft texture, and adequate elasticity, the animal feeds are suitable for serving as the food for the companion animals that have weaker teeth; including puppies, older dogs, and cats. For packing, adequate amounts of the aforesaid animal feeds should be packed into packages, followed by sealing in order to make the animal feeds into final products. Said packages are preferably airtight packing materials that do not allow oxygen to pass through. Methods for packing include vacuum packing and packing in packages filled with active carbon, but the preferable method is to use packages simultaneously filled with deoxidants (For example, Ageless™) and active carbon. This packing method or similar ones protects the animal feeds from spoiling resulted from oxidation and microbial growth within an expiry date.

The testosterone generating and metabolizing enhancer of the invention may also be used on skin externally (such as in cosmetics, medicines, and external medical applications), and the substances for external uses on skin may inhibit the actions of hyaluronidase. The substances for external uses on skin may be applied to humans, and may also be applied to mammals other than the humans. The testosterone generating and metabolizing enhancer may be applied in substances for external uses on skin, which includes lotions, bars of soap, facial cleansers, body cleansers, skin creams, lotions, toners, toning lotions, shaving creams, shaving toners, makeup oils, sun-blocking toners, foundation powder, foundation, perfumes, masks, nail polishes, enamels, nail polish removers, brow dyes, blushes, eye creams, eye shadows, mascaras, eyeliners, lipsticks, lip glosses, shampoos, hair conditioners, hair dyes, dissipating solutions, and washing solutions. In addition, the testosterone generating and metabolizing enhancer may be applied as medicines or for external medical uses in forms of ointments, creams, and external-use liquids.

In addition to the testosterone generating and metabolizing enhancer, the aforesaid substances for external uses on skin may also have the following substances, which do not impede the generation and metabolism of testosterone and may also be added into cosmetics and medicines for external uses on skin; including: oil, higher alcohol, fatty acid, ultraviolet absorber, powder, colorant, surfactant, polyhydric alcohol, sugar, polymer, biologically active ingredient, solvent, anti-oxidant, aroma chemical (perfume material), and preservatives. Examples of the aforesaid substances are listed below, though the invention is not limited to these examples.
(1) Specific examples of oil, particularly the ester-type oil phase ingredients including: triglyceryl 2-ethylhexanoate, cetyl 2-ethylhexanoate, isopropyl myristate, butyl myristate, isopropyl palmilate, ethyl stearate, octyl palmitate, isocetyl isostearate, butyl stearate, butyl myristate, ethyl linoleate, isopropyl linoleate, ethyl oleate, isocetyl myristate, isostearyl myristate, isostearyl palmitate, octyldodecyl myristate, isocetyl isostearate, diethyl sebacate, diisopropyl adipate, isoarachyl neopentanoate, caprylic-capric acid triglyceride, trimethylolpropane tri-2-ethylhexrattoate, trimethylolpropane triisostearate, pentaerythritol tetra-2-ethylhexanoate, cetyl caprylate, decyl laurate, hexyl laurate, decyl myristate, myristyl myristate, cetyl myristate, stearyl stearate, decyl oleate, cetyl ricinoleate, isostearyl laurate, isotridecyl myristate, isocetyl myristate, isostearyl myristate, isocetyl palmitate, isostearyl palmitate, octyl stearate, isocetyl stearate, isodecyl oleate, octyldodecyl oleate, octyldodecyl linoleate, isopropyl isostearate, cetostearyl 2-ethylhexanoate, stearyl 2-ethylhexanoate, hexyl isostearate, ethylene glycol dioctanoate, ethylene glycol dioleate, propylene glycol dicaprate, propylene glycol di (caprylate/caprate), propylene glycol dicaprylate, neopentyl glycol dicaprate, neopentyl glycol dioctanoate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerin oleate, polyglycerin isostearate, dipropyl carbonate, dialkyl carbonate (C12-18), triisocetyl citrate, triisoarachyl citrate, triisooctyl citrate, lauryl lactate, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoylhydroxystearate, stearyl 12-stearoylhydroxystearate and isostearyl 12-stearoylhydroxystearate. The oil may also include hydrocarbon-type oil phase ingredients such as: squalane, liquid paraffin, a-olefin oligomer, isoparaffin, ceresin, paraffin, liquid isoparaffin, polybutene, microcrystalline wax and Vaseline. The oil may also include animal and plant oils, hardened oils thereof, and wax of natural origin such as: beef tallow, hardened beef tallow, lard, hardened lard, horse oil, hardened horse oil, mink oil, orange roughy oil, fish oil, hardened fish oil and egg yolk oil; plant oils and hardened oils thereof such as: avocado oil, almond oil, olive oil, cocoa oil, apricot kernel oil, kukui nut oil, Aloe arborescens oil, sesame oil, wheat germ oil, rice germ oil, rice bran oil, safflower oil, shea butter, soybean oil, evening primrose oil, perilla oil, tea seed oil, tsubaki oil (camellia japonica oil), corn oil, rapeseed oil, hardened rapeseed oil, palm kernel oil, hardened palm kernel oil, peanut oil, hardened peanut oil, castor oil, hydrogenated castor oil, sunflower oil, grape seed oil, jojoba oil, hardened jojoba oil, macadamia nut oil, meadowfoam seed oil, cottonseed oil, hardened cottonseed oil, conoanut oil, hardened cocoanut oil; and waxes such as beeswax, high acid number beeswax, lanolin, reduced lanolin, hardened lanolin, liquid lanolin, carnauba wax and montan wax. The oil may also include silicone-type oil phase ingredients such as: dimethylpolysiloxane, methylphenylpolysiloxane, methylcyclopolysiloxane, octamethylpolysiloxane, decamethylpolysiloxane, dodecamethylcyclosiloxane, methylhydrogenpolysiloxane, polyether-modified organopolysiloxane, dimethylsiloxanemethylcetyloxysiloxane copolymer, dimethylsiloxane-methylstearoxysiloxane copolymer, alkyl-modified organopolysiloxane, terminal-modified organopolysiloxane, amino-modified silicone oil, amino-modified organopolysiloxane, dimethiconol, silicone gel, acryl silicone, trimethylsiloxysilicic acid and silicone RTV rubber. The oil may also include fluorine-type oil phase ingredients such as: perfluoropolyether, fluoro-modified organopolysiloxane, fluorinated pitch, carbon fluoride, fluorinated alcohols, fluorinated alkyl, and polyhydroxy alkylene modified organopolysiloxane.
(2) Specific examples of higher alcohol including: lauryl alcohol, myristyl alcohol, cetyl alcohol, stearyl alcohol, isostearyl alcohol, oleyl alcohol, behenyl alcohol, 2-ethylhexanol, hexadecyl alcohol, and octyl dodecanol.
(3) Specific examples of fatty acid including: caprylic acid, capric acid, undecylenic acid, lauric acid, myristic acid, palmitic acid, palmitoleic acid, stearic acid, isostearic acid, oleic acid, linoleic acid, linolenic acid, arachic acid, arachidonic acid, behenic acid, erucic acid and 2-ethylhexanoic acid.
(4) Specific examples of ultraviolet absorber including: para-aminobenzoic acid, amyl para-aminobenzoate, ethyldihydroxypropyl para-aminobenzoate, glyceryl para-aminobenzoate, ethyl para-aminobenzoate, octyl para-aminobenzoate, octyldimethyl para-aminobenzoate, ethylene glycol salicylate, octyl salicylate, triethanolamine salicylate, phenyl salicylate, butylphenyl salicylate, benzyl salicylate, homomethyl salicylate, benzyl cinnamate, octyl para-methoxycinnamate, 2-ethylhexyl para-methoxycinnamate, glyceryl mono-2-ethyl hexanoate di-para-methoxycinnamate, isopropyl para-methoxycinnamate, diethanolamine para-methoxyhydrocinnamate, diisopropyl diisopropylcinnamic acid ester mixture, urocanic acid, ethyl urocanate, hydroxymethoxybenzophenone, hydroxymethoxybenzophenone sulfonic acid and a salt thereof, dihydroxymethoxybenzophenone, sodium dihydroxymethoxybenzophenonedisulfonate, dihydroxybenzophenone, dihydroxydimethoxybenzophenone, hydroxyoctoxybenzophenone, tetrahydroxybenzophenone, butylmethoxydibenzoylmethane, 2,4, 6-trianilino-p-(carbo-2-ethylhexyl-1-oxy)-1,3,5-triazine, 2-(2-hydroxy-5-methylphenyl)benzotriazole, methyl-O-aminobenzoate, 2-ethylhexyl-2-cyano-3,3-diphenylacrylate, phenylbenzimidazole sulfuric acid, 3-(4-methylbenzylidene)camphor, isopropyldibenzoylmethane, 4-(3, 4-dimethoxyphenylmethylene)-2,5-doxy-1-imidazolidinepropionate, and polymer derivatives and silane derivatives thereof.
(5) Specific examples of powder and pigment including: pigments such as Food Red 104, Food Red 201, Food Yellow 4, Food Blue 1 and Food Black 401; lake pigments such as Food Yellow 4 AL lake and Food Yellow 203 BA lake; polymers such as nylon powder, silk powder, urethane powder, Teflon® powder, silicone powder, polymethyl methacrylate powder, cellulose powder, starch, silicone elastomer spherical powder and polyethylene powder; color pigments such as yellow iron oxide, red iron oxide, black iron oxide, chromium oxide, carbon black, ultramarine and iron blue; white pigments such as zinc oxide, titanium oxide and cerium oxide; extender pigments such as talc, mica, sericite, kaolin and plate barium sulfate; pearl pigments such as mica titanium; metal salts such as barium sulfate, calcium carbonate, magnesium carbonate, aluminum silicate and magnesium silicate; inorganic powders such as silica and alumina; metal soaps such as aluminum stearate, magnesium stearate, zinc palmitate, zinc myristate, magnesium myristate, zinc laurate and zinc undecylenate; bentonite; smectite; and boron nitride. The shape (e.g., sphere, bar, needle, plate, amorphous, scale, spindle) and the particle size of these powders are not particularly limited. Moreover, these powders may or may not be previously surface-treated by using a conventionally known surface treatment (such as fluorine compound treatment, silicone treatment, silicone resin treatment, pendant treatment, saline coupling agent treatment, titanium coupling agent treatment, lubricant treatment, N-acylated lysine treatment, polyacrylic acid treatment, metal soap treatment, amino acid treatment, lecithin treatment, inorganic compound treatment, plasma treatment, and mechanochemical treatment).
(6) Specific examples of surfactant including: anionic surfactants like fatty acid soap, a-acyl sulfonate, alkyl sulfonate, alkylallyl sulfonate, alkylnaphthalene sulfonate, alkyl sulfate, POE alkyl ether sulfate, alkylamide sulfate, alkyl phosphate, POE alkyl phosphate, alkylamide phosphate, alkyloylalkyl taurine salt, N-acylamino acid salt, POE alkyl ether carbonate, alkyl sulfosuccinate, sodium alkylsulfoacetate, acylated hydrolyzed collagen peptide salt, and perfluoroalkylphosphoric acid ester. The surfactant may also include cationic surfactants such as: alkyltrimethylammonium chloride, stearyltrimethylammonium chloride, stearyltrimethylammonium bromide, cetostearyltrimethylammonium chloride, distearyldimethylammonium chloride, stearyldimethylbenzylammonium chloride, behenyltrimethylammonium bromide, benzalkonium chloride, behenic acid amidopropyldimethyl hydroxypropylammonium chloride, diethylaminoethylamide stearate, dimethylaminoethylamide stearate, dimethylaminopropylamide stearate and lanolin derivative quaternary ammonium salt. The surfactant may also include amphoteric surfactants such as: carboxybetaine type, amidobetaine type, sulfobetaine type, hydroxysulfobetaine type, amidosulfobetaine type, phosphobetaine type, aminocarboxylate type, imidazoline derivative type and amidoamine type. The surfactant may also include non-ionic surfactants such as: propylene glycol fatty acid ester, glycerin fatty acid ester, polyglycerin fatty acid ester, sorbitan fatty acid ester, POE sorbitan fatty acid ester, POE sorbitol fatty acid ester, POE glycerin fatty acid ester, POE alkyl ether, POE fatty acid ester, POE hydrogenated castor oil, POE castor oil, POE-POP copolymer, POE-POP alkyl ether, polyether-modified silicone lauric acid alkanolamide, alkylamine oxide and hydrogenated soybean phospholipid. The surfactant may also include natural-type surfactants such as: lecithin, saponin, and sugar-type surfactant.
(7) Specific examples of polyhydric alcohol and sugar including: ethylene glycol, diethylene glycol, polyethylene glycol, propylene glycol, dipropylene glycol, polypropylene glycol, glycerin, diglycerin, polyglycerin, 3-methyl-1, 3-butanediol, 1,3-butylene glycol, sorbitol, mannitol, raffinose, erythritol, glucose, sucrose, fructose, xylitol, lactose, maltose, maltitol, trehalose, alkylated trehalose, mixed isomerized sugar, sulfated trehalose, and pullulan. Chemically modified products thereof can also be used.
(8) Specific examples of polymer including: anionic polymers such as acrylic acid ester/methacrylic acid ester copolymer (PLUS-SIZE, produced by Sogokagaku K. K.), vinyl acetate/crotonic acid copolymer (Resin 28-1310, produced by NSC), vinyl acetate/crotonic acid/vinyl neodecanate copolymer (28-2930, produced by NSC), methyl vinyl ether maleic acid half ester (GANTREZ ES, produced by ISP), T-butyl acrylate/ethyl acrylate/methacrylic acid copolymer (RUBIMER, produced by BASF), vinylpyrrolidone/vinyl acetate/vinyl propionate copolymer (RUBISCOL VAP, produced by BASF), vinyl acetate/crotonic acid copolymer (RUBISET CA, produced by BASF), vinyl acetate/crotonic acid/vinylpyrrolidone copolymer (RUBISET CAP, produced by BASF), vinylpyrrolidone/acrylate copolymer (RUBIFLEX, produced by BASF), acrylate/acrylamide copolymer (ULTRAHOLD, produced by BASF), vinyl acetate/butyl maleate-isobornyl acrylate copolymer (ADVANTAGE, produced by ISP), carboxy vinyl polymer (CARBOPOL, produced by BF Goodrich), and acrylic acid-alkyl methacrylate copolymer (PAMUREN, produced by BF Goodrich). The polymer may also include amphoteric polymers such as: acetic acid amphoteric compound of dialkylaminoethyl methacrylate polymer (YUKAFORMER, produced by Mitsubishi Chemical) and octylacrylamide acrylate/hydroxypropyl acrylate/butylaminoethyl methacrylate copolymer (AMPHOMER, produced by NSC). The polymer may also include cationic polymers such as: quaternized compound of vinylpyrrolidone/dimethylaminoethyl methacrylate (GAFQUAT, produced by ISP) and methyl vinyl imidazolium chloride/vinylpyrrolidone copolymer (RUBICOTE, produced by BASF). The polymer may also include non-ionic polymers such as: polymer compounds such as polyvinylpyrrolidone (RUBISCOL K, produced by BASF), vinylpyrrolidone/vinyl acetate copolymer (RUBISCOL VA, produced by BASF), vinylpyrrolidone/dimethylaminoethyl methacrylate copolymer (COPOLYMER 937, produced by ISP), and vinylcaprolactam/vinylpyrrolidone/dimethylaminoethyl methacrylate copolymer (COPOLYMER VC713, produced by ISP). The polymer may also include natural-type polymers such as: cellulose and derivatives thereof, keratin and collagen and derivatives thereof, calcium alginate, pullulan, agar, gelatin, tamarind seed polysaccharides, xanthane gum, carrageenan, high-methoxyl pectin, low-methoxyl pectin, guar gum, Arabia gum, crystal cellulose, arabino galactan, karaya gum, tragacanth gum, alginic acid, albumin, casein, cardrun, gellan gum and dextran.
(9) Specific examples of biologically active ingredient, which may include substances that are capable of imparting some biological activities to skin when being applied to the skin, such as: whitening ingredient, anti-inflammatory agent, anti-aging ingredient, ultraviolet-resistance ingredient, slimming agent, skin tightening agent, antioxidant, hair restorer, hair growing agent, moisturizer, blood circulation accelerator, antibacterial agent, bactericide, desiccant, cooling agent, warming agent, vitamin compound, amino acid, wound healing accelerator, torpent, analgetic, cell activator, and enzyme. Suitable ingredients may be blended therewith, including: angelica extract, avocado extract, hydrangea extract, althea extract, arnica extract, aloe extract, apricot extract, apricot core extract, ginkgo extract, fennel extract, turmeric extract, oolong tea extract, rose fruit extract, echinacea leaf extract, scutellaria root extract, phellodendron bark extract, goldthread extract, barley extract, hypericum extract, white nettle extract, watercress extract, orange extract, sea salt, seaweed extract, hydrolyzed elastin, hydrolyzed wheat powder, hydrolyzed silk, chamomile extract, carrot extract, artemisia capillaris extract, glycyrrhiza extract, sabdariffa extract, pyracantha fortuneana fruit extract, cinchona extract, cucumber extract, guanosine, gardenia extract, sasa albo-marginata extract, sophora root extract, walnut extract, grapefruit extract, clematis extract, chlorella extract, mulberry bark extract, gentian extract, black tea extract, yeast extract, burdock extract, fermented rice bran extract, rice germ oil, comfrey extract, collagen, cowberry extract, asiasarum root extract, bupleurum falcatum root extract, umbilical cord extract, salvia extract, saponaria extract, bamboo grass extract, crataegus extract, zanthoxylum fruit extract, shiitake mushroom extract, rehmannia root extract, lithospermum root extract, perilla extract, linden extract, filipendula extract, peony root extract, calamus rhizome extract, birch extract, horsetail extract, ivy extract, hawthorn extract, sambucus nigra extract, yarrow extract, peppermint extract, sage extract, mallow extract, enidium rhizome extract, swertia herb extract, soy extract, jujube extract, wild thyme extract, green tea extract, clove extract, cogon extract, citrus unshiu peel extract, angelica root extract, calendula extract, peach seed extract, bitter orange extract, houttuynia extract, tomato extract, natto extract, ginseng extract, garlic extract, wild rose extract, hibiscus sabdariffa flower extract, ophiopogon tuber extract, parsley extract, honey, witch hazel extract, pellitory extract, isodonis extract, matricaria extract, loquat extract, coltsfoot extract, butterbur scape extract, Poria cocos extract, butcher bloom extract, grape extract, propolis, luffa extract, safflower extract, peppermint extract, linden extract, peony extract, hop extract, pine extract, horse chestnut extract, skunk cabbage extract, sapindaceae extract, balm mint extract, peach extract, cornflower extract, eucalyptus extract, saxifrage extract, coix seed extract, mugwort extract, lavender extract, apple extract, lettuce extract, lemon extract, Chinese milk vetch extract, rose extract, rosemary extract, Roman chamomile extract, and royal jelly extract. Other suitable ingredients may be blended therewith, including biopolymers such as: deoxyribonucleic acid, mucopolysaccharide, sodium hyaluronate, sodium chondroitin sulfate, collagen, elastin, chitin, chitosan, and hydrolyzed eggshell membrane. Other suitable ingredients may be blended therewith, including moisture retentive ingredients such as: amino acid, hydrolyzed peptide, sodium lactate, urea, sodium pyrrolidonecarboxylate, betaine, whey, and trimethylglycine. Other suitable ingredients may be blended therewith, including oil-type ingredients such as: sphingolipid, ceramide, phytosphingosine, cholesterol, cholesterol derivatives, and phospholipid. Other suitable ingredients may be blended therewith, including anti-inflammatory agents such as: ε-aminocaproic acid, glycyrrhizic acid, β-glycyrrhetic acid, lysozyme chloride, guaiazlene, and hydrocortisone. Other suitable ingredients may be blended therewith, including vitamins such as: vitamin A, vitamin B2, vitamin B6, vitamin C, vitamin D, vitamin E, calcium pantothenate, biotin, nicotinic acid amide, and vitamin C ester. Other suitable ingredients may be blended therewith, including active ingredients such as: allantoin, diisopropylamine dichloroacetate, and 4-aminomethylcyclohexanecarboxylic acid. Other suitable ingredients may be blended therewith, including anti-oxidants such as: tocopherol, carotenoid, flavonoid, tannin, lignin and saponin. Other suitable ingredients may be blended therewith, including cell activators such as: α-hydroxy acid and β-hydroxy acid. Other suitable ingredients may be blended therewith, including blood circulation accelerators such as γ-orizanol and vitamin E derivatives. Other suitable ingredients may be blended therewith, including wound healing accelerators such as retinol and retinol derivatives. Other suitable ingredients may be blended therewith, including whitening ingredients such as: albumin, kojic acid, placenta extract, sulfur, ellagic acid, linoleic acid, tranexamic acid, and glutathione. Other suitable ingredients may be blended therewith, including hair growing agents such as: cepharanthine, glycyrrhiza extract, capsicum tincture, hinokitiol, iodized garlic extract, pyridoxine hydrochloride, DL-α-tocopherol, DL-α-tocopheryl acetate, nicotinic acid, nicotinic acid derivatives, calcium pantothenate, D-pantothenyl alcohol, acetyl pantothenylethyl ether, biotin, allantoin, isopropylmethylphenol, estradiol, ethynyl estradiol, capronium chloride, benzalkonium chloride, diphenhydramine hydrochloride, Takanal, camphor, salicylic acid, vanillylamide nonylate, vanillylamide nonanoate, pyroctone olamine, glyceryl pentadecanoate, L-menthol, mononitroguaiacol, resorcinol, γ-aminobutyric acid, benzethonium chloride, mexiletine hydrochloride, auxin, female hormone, cantharis tincture, cyclosporine, zinc pyrithione, hydrocortisone, minoaxidil, polyoxyethylene sorbitan monostearate, peppermint oil, and SADANISHIKI extract.
(10) Specific examples of anti-oxidant including: sodium hydrogensulfite, sodium sulfite, erythorbic acid, sodium erythorbate, dilauryl thiodipropionate, tocopherol, tolylbiguanide, nordihydroguaiaretic acid, parahydroxy anisole, butylhydroxy anisolc, dibutylhydroxy toluene, ascorbyl stearate, ascorbyl palmitate, octyl gallate, propyl gallate, carotenoid, flavonoid, tannin, lignin, saponin, and plant extracts having antioxidant effect, such as apple extract and clove extract.
(11) Specific examples of solvent including: purified water, ethanol, lower alcohol, ethers, LPG, fluorocarbon, N-methylpyrrolidone, fluoroalcohol, volatile linear silicone, and next generation fleon.

### EXAMPLES

Although particular examples of the invention will be described in details for purposes of illustration, the invention is not limited to the following examples.

### Example

Preparation of the testosterone generating and metabolizing enhancer of the present invention.

### Example 1

Preparation of extracts from the holoparasitic plant Cistanche tubulosa of the Cistanche family (abbreviated as C. tubulosa hereafter).

Methods for Extraction

10 kg of thinly sliced fresh stems of C. tubulosa were immersed in water that was eight times the weight of the thin slices for one hour, and then boiled for two hours; the boiled mixture was subsequently filtered to obtain a first filtered solution. Afterwards, leftovers from the first filtration were mixed with 40% ethanol that was four times the weight of the leftovers and boiled for four hours; the boiled mixture was filtered again to obtain a second filtered solution. Subsequently, leftovers from the second filtration were subjected to the aforesaid procedures for another two times in order to obtain a third filtered solution and a fourth filtered solution. The aforesaid four filtered solutions were then combined and concentrated in vacuum until a resulted concentrated solution had reached a weight of 1.05 (50° C.), thereby resulting in a final extract. Said final extract obtained by using the aforesaid method had a weight of 6.2 kg.

Methods for Purification

6 kg of the aforesaid final extract was dissolved in water that was half the weight of the final extract by heating, and then a resulted solution having the final extract was allowed to pass through the aforesaid adsorption columns filled with Type D-101 large-hole adsorption resins. Afterwards, water was firstly used to elute the adsorption columns, and a water eluting liquid that was twice the weight of said fresh stems was obtained; 20% ethanol was subsequently used to elute the adsorption columns and obtain a first eluting liquid of 20% ethanol that was also twice the weight of said fresh stems. Subsequently, said water eluting liquid underwent one more round of adsorption-elution steps, and a second eluting liquid of 20% ethanol was obtained. The two 20% ethanol eluting liquids were consequently combined, concentrated, and dried to obtain an extract having phenylethanoid glycosides (the first embodiment). The extract had a weight of 0.865 kg.

The concentrations of echinacoside and acteoside were determined by using HPLC, and the HPLC was set as follows: the stationary phase was based on stearyl-silance bonded silica gel; the mobile phase was based on methanol-0.15% acetic acid solution (30:70), and the flow rate was 1 ml/min; the detection wavelength was set at 330 nm.

After drying in vacuum for 24 hours at 60° C., the echinacoside and acteoside were dissolved in 50% methanol in order to make a control solution (in which 1 ml of solution has 0.1 mg of dissolved substances).

Using supersonic treatment, 50 mg of the extract having phenylethanoid glycosides was dissolved in 50% methanol of adequate amount in a 25 ml container, and then more 50% methanol was added into said solution until said solution reached a line indicating 25 ml. Precisely obtained 1 ml of said solution to place into a 10 ml container, and filled 50% methanol into the container until a line indicating 10 ml was reached. Said solution was filtered by using a 0.45 μm membrane to obtain a sample solution.

5 μl was separately obtained from the control solution and the sample solution and injected into a HPLC instrument, so as to respectively determine peak areas of echinacoside and acteoside, so that concentrations of echinacoside and acteoside could be calculated from the peak areas thereof. The outcome was that echinacoside occupied 29.3 wt % in the extract (the first embodiment), and acteoside took up 10.0 wt % in the extract (the first embodiment).

### Examples 2 and 3

A pure product of echinacoside was used in Example 2 (manufactured by Sinphar Pharmaceutical Co., Ltd.), while a pure product of acteoside was used in Example 3 (manufactured by Sinphar Pharmaceutical Co., Ltd.).

Test 1: Testing testosterone generation of the mouse Leydig tumor cell line R2C.

Materials: The extract of C. tubulosa prepared in the aforesaid first embodiment, and said echinacoside and acteoside used in the second and the third embodiments. The mouse Leydig tumor cell line R2C was obtained from the Japan Health Sciences Foundation (Osaka), and a Testosterone EIA testing kit was used to test testosterone levels.

Method: The R2C cells were subcultured in a cell medium called Ham's F10, which comprised 15% bovine serum, 2.5% fetal bovine serum, 100 units/mL penicillin G, and 100 μg/mL streptomycin. The cells were inoculated on a 24-hole plate at a density of 5×10⁵ cells/mL, and 500 μL of the cell medium was added into each hole. After culturing for 24 hours, 50 μL of solutions resulted from dissolving samples from the first to the third embodiments in the same cell medium was added into each hole. After culturing for another 4 fours, the cells were collected along with the cell medium and subjected to centrifugation, so as to collect supernatant that included the cell medium. The Testosterone EIA testing kit (manufactured by Cayman Chemical Corporate) was used to determine testosterone concentrations in the each tested sample, and the results were shown in FIG. 1.

Results and Effects of Examples in Test 1: As shown in FIG. 1, the testosterone generation was reduced in cells that received 10 μg/mL and 30 μg/mL of the C. tubulosa extract (the first embodiment). However, in cells that received 100 μg/mL of the C. tubulosa extract, the testosterone level in the supernatant thereof was significantly higher. The result indicated that the C. tubulosa extract from the first embodiment helps enhance the generation of testosterone. On the other hand, when viewing the result from the perspective of chemical components, no matter echinacoside (the second embodiment) was applied at 10 μg/mL, 30 μg/mL, or 100 μg/mL; the testosterone generation in the samples was increased accordingly, and especially when echinacoside was applied at the concentration of 100 μg/mL, under which the testosterone generation was increased to twice as much as that of the control (Cont.). Therefore, the result suggested that echinacoside was effective for enhancing testosterone generation. In addition, testosterone generation was also found to have increased when acteoside (the third embodiment) was applied at both 10 μg/mL and 100 μg/mL.

Test 2: Testing testosterone levels in the bloodstream of mouse.

Method: 5-month-old male ddY mice were orally fed the C. tubulosa extract from the aforesaid first embodiment, and the regimen continued for two weeks. Afterwards, blood samples were collected from the mice in order to determine the testosterone levels therein, and blood samples were also collected from 6-week-old mice to serve as a control.

Results and Effects of Examples in Test 2: As revealed in Table 2, the feeding of the C. tubulosa extract (200 mg/kg and 400 mg/kg) increased levels of testosterone in the bloodstream of the mice.

TABLE 2
Effects of the C. tubulosa extract on the testosterone
level in the bloodstream of the mice

| | | | Testosterone |
|---|---|---|---|
| | Dosage (mg/kg) | N | (ng/mL) |
| 6-week-old mice | — | 4 | 8.98 ± 1.97 |
| 5-month-old mice | — | 6 | 29.53 ± 10.4 |
| the C. tubulosa extract | 200 | 7 | 40.41 ± 29.64 |
| | 400 | 7 | 79.11 ± 44.66 |

mean ± S.D.

Test 3: Effects on the expression of 5α-reductase in mice livers (microarray analysis)

Method: Firstly chose a 5-month-old mouse (from the control) from the aforesaid Test 2 and a mouse that was fed the C. tubulosa extract of the first embodiment only, and then removed the livers from the mice. Subsequently, thin slices of the livers (approximately 10 mg) were immersed in RNAlater (manufactured by Qiagen K. K.) for stabilization, and then Rneasy protect mini (manufactured by Qiagen K. K.) was used to extract and purify total RNA. This was followed by the use of genetic chips (Mouse 4302.0, manufactured by Affymetrix Inc.) to carry out microarray analysis for gene expression of the RNA; the result is shown below.

TABLE 3
Effects of the C. tubulosa extract on the gene
expression of 5α-reductase in mice livers

| | Expression Ratio |
|---|---|
| Gene | (the C. tubulosa extract group/control) |
| 5α-reductase (Srd5a2) | 2.23 |

Results and Effects of Examples in Test 3: Referring to Table 3; in the liver of the mouse being fed the C. tubulosa extract (400 mg/kg) of the first embodiment, the gene expression of 5α-reductase was increased 2.24-fold as compared to the control. The result indicated that the C. tubulosa extract of the first embodiment was effective for enhancing the gene expression of 5α-reductase, which in turn enhanced the metabolism of testosterone into dihydrotestosterone.

Test 4: Testing effects of repeated feeding of the extract of the first embodiment on the gene expression of enzymes responsible for testosterone synthesis and metabolism (by using RT-PCR).

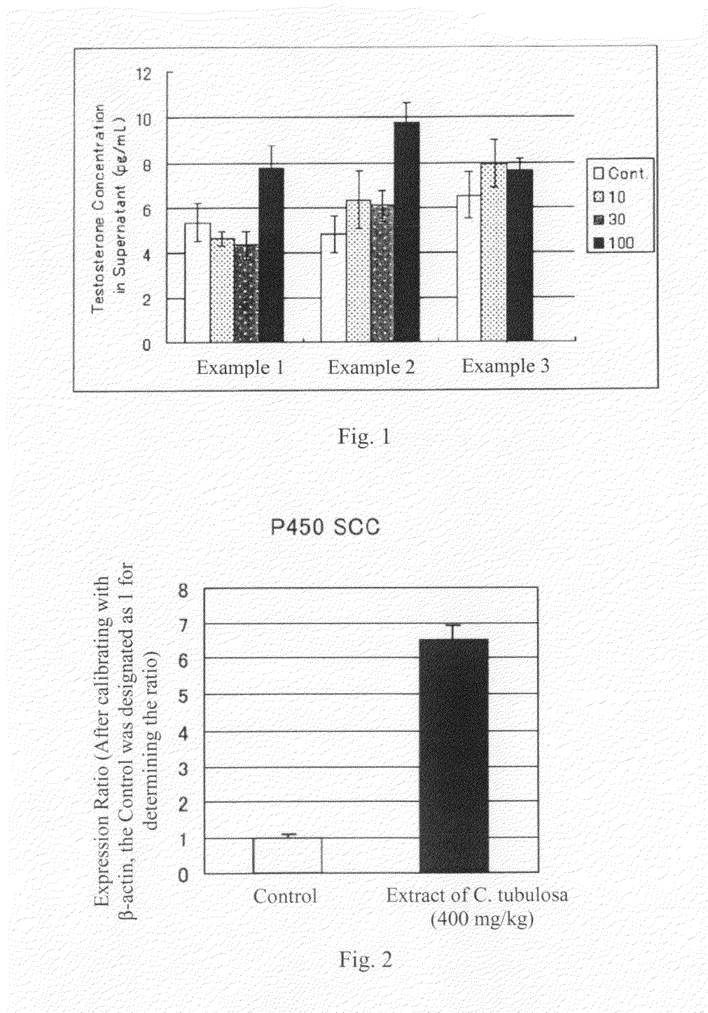

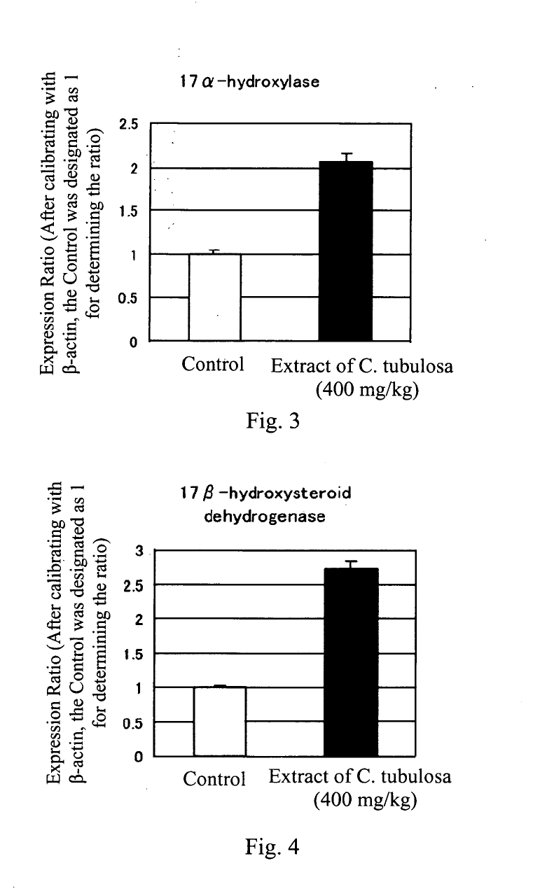

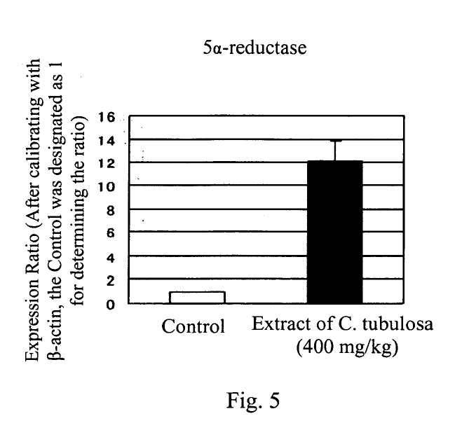

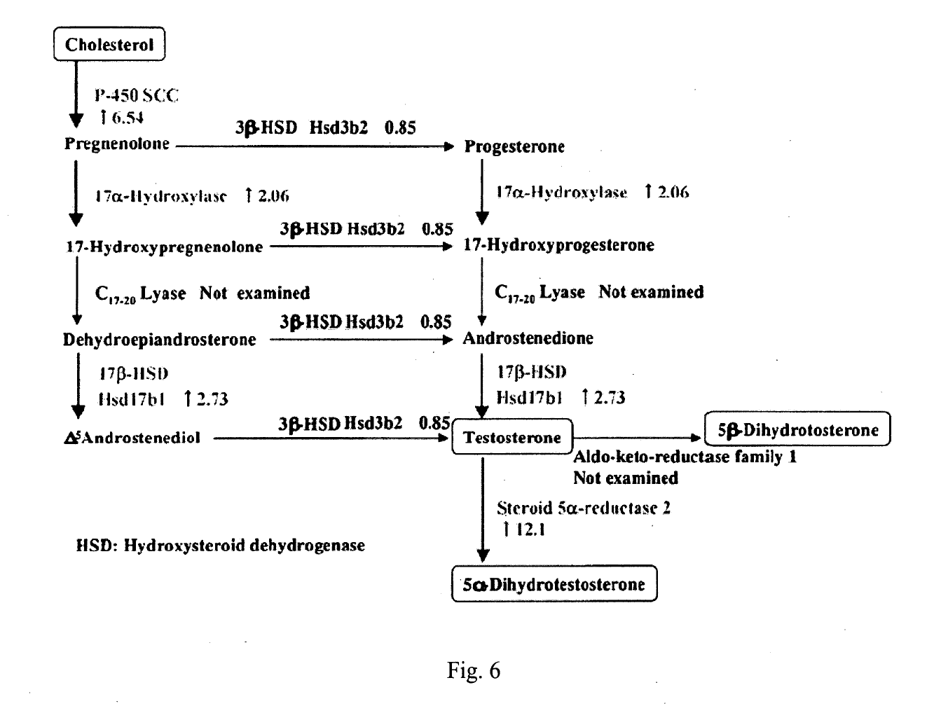

Method: 5-month-old male ddY mice were orally fed 400 mg/kg of the C. tubulosa extract (the extract of the first embodiment), and the regimen continued for two weeks before livers were removed from the mice; RNAlater was then used to stabilize the livers, and RNA was extracted from the livers by using a testing kit manufactured by Qiagen K. K. Using commonly available methods, c-DNA was fabricated via reverse transcription, and gene expression was determined via RT-PCR. The results are shown in FIG. 2 (P450 SCC), FIG. 3 (17α-hydroxylase), FIG. 4 (17β-hydroxysteroid dehydrogenase), and FIG. 5 (5α-reductase). In addition, FIG. 6 is a flow chart that shows the relationship between the enzymes indicated in FIGS. 2-5, in which the pathway for synthesizing testosterone from cholesterol in the liver was illustrated, as well as the pathway for metabolizing testosterone into dihydrotestosterone. In FIG. 6, the figures shown on the right side of the name of the enzymes were the scale of the gene expression relative to the control, and the upward-pointing arrows represented an increase in the gene expression of the enzyme.

Results and Effects of the Embodiments in Test 4: The results shown in FIGS. 2-4 and FIG. 6 suggested that the extract of the first embodiment was effective for enhancing the gene expression of P450 SCC (side chain cleavage) enzyme, 17α-hydroxylase, and 17β-hydroxysteroid dehydrogenase, and these enzymes are related to the synthesis of testosterone from cholesterol. In other words, the extract of the first embodiment was effective for increasing the testosterone level in the bloodstream by enhancing the gene expression of said enzymes. Moreover, the results shown in FIGS. 5 and 6 indicated that the extract of the first embodiment was also effective for enhancing the gene expression of 5α-reductase; the enzyme responsible for metabolizing testosterone into dihydrotestosterone. As a result, the extract of the first embodiment was confirmed to be effective for enhancing the metabolism of testosterone.

Formulas for formulating the testosterone generating and metabolizing enhancer of the invention are listed below, though the invention is not limited to these formulas.

Formula 1: for a chewing gum

| Sugar | 53.0 | wt % |
|---|---|---|
| Gum base | 20.0 | |
| Glucose | 10.0 | |
| Malt sugar | 16.0 | |
| Aroma chemicals | 0.5 | |
| The testosterone generating and | 0.5 | |
| metabolizing enhancer | | |
| | 100.0 | wt % |

Formula 2: for a chewing sweet

| Maltitol | 40.0 | wt % |
|---|---|---|
| Sugar | 20.0 | |
| Glucose | 20.0 | |
| Gelatin | 4.7 | |
| Water | 9.68 | |
| Grape juice | 4.0 | |
| Grape flavor | 0.6 | |
| Colorants | 0.02 | |
| The testosterone generating and | 1.0 | |
| metabolizing enhancer | | |
| | 100.0 | wt % |

Formula 3: for a candy

| Sugar | 50.0 | wt % |
|---|---|---|
| Malt sugar | 33.0 | |
| Water | 14.4 | |
| Organic acids | 2.0 | |
| Aroma chemicals | 0.2 | |
| The testosterone generating and | 0.4 | |
| metabolizing enhancer | | |
| | 100.0 | wt % |

Formula 4: for a yoghurt (hard or soft)

| Milk | 41.5 | wt % |
|---|---|---|
| Non-fat milk powder | 5.8 | |
| Sugar | 8.0 | |
| Agar | 0.15 | |
| Gelatin | 0.1 | |
| Lactic acid bacteria | 0.005 | |
| The testosterone generating and | 0.4 | |
| metabolizing enhancer | | |
| Aroma chemicals | Minute amount | |
| Water | For making the product | |
| | up to 100 wt % | |
| | 100.0 | wt % |

Formula 5: for a cooling drink

| Solutions of fructose and glucose | 30.0 | wt % |
|---|---|---|
| Emulsifier | 0.5 | |
| The testosterone generating and | 0.05 | |
| metabolizing enhancer | | |
| Aroma chemicals | Appropriate amounts | |
| Purified water | For making the product | |
| | up to 100 wt % | |
| | 100.0 | wt % |

Formula 6: for a dessert

| Sugar | 76.4 | wt % |
|---|---|---|
| Glucose | 19.0 | |
| Sucrose esters | 0.050.2 | |
| The testosterone generating and | 0.5 | |
| metabolizing enhancer | | |
| Purified water | 3.9 | |
| | 100.0 | wt % |

Formula 7: for a soft capsule

| Grape seed oil | 87.0 | wt % |
|---|---|---|
| Emulsifier | 12.0 | |
| The testosterone generating and | 1.0 | |
| metabolizing enhancer | | |
| | 100.0 | wt % |

Formula 8: for a dose in the form of slices

| Lactose | 54.0 | wt % |
|---|---|---|
| Crystal cellulose | 30.0 | |
| Degradable starch | 10.0 | |
| Glycerin fatty acid esters | 5.0 | |
| The testosterone generating and | 1.0 | |
| metabolizing enhancer | | |
| | 100.0 | wt % |

Formula 9: for a dose of oral
granules (a pharmaceutical product)

| The testosterone generating and | 1.0 | wt % |
|---|---|---|
| metabolizing enhancer | | |
| Lactose | 30.0 | |
| Corn starch | 60.0 | |
| Crystal cellulose | 8.0 | |
| Polyvinyl pyrrolidone | 1.0 | |
| | 100.0 | wt % |

Formula 10: for a cosmetic paste

| Squalane | 20.0 | wt % |
|---|---|---|
| Bees' wax | 5.0 | |
| Top jojoba oil | 5.0 | |
| Glycerin | 5.0 | |
| Glycerol monostearate | 2.0 | |
| Polyoxyethylene (20) sorbitan | 2.0 | |
| monostearate | | |
| The testosterone generating and | 2.0 | |
| metabolizing enhancer | | |
| Preservatives | Appropriate amounts | |
| Aroma chemicals | Appropriate amounts | |
| Purified water | For making the product | |
| | up to 100 wt % | |
| | 100.0 | wt % |

Formula 11: for a toner

| Ethanol | 5.0 | wt % |
|---|---|---|
| Glycerin | 2.0 | |
| 1,3-butylene glycol | 2.0 | |
| Polyethylene oil ether | 0.5 | |
| Sodium citrate | 0.1 | |
| Citric acid | 0.1 | |
| The testosterone generating and | 0.1 | |
| metabolizing enhancer | | |
| Purified water | For making the product | |
| | up to 100 wt % | |
| | 100.0 | wt % |

Formula 12: for a body lotion

| Macadamia nut oil | 2.0 | wt % |
|---|---|---|
| Octyldodecyl myristate | 10.0 | |
| Mehtyl phenyl polysiloxane | 5.0 | |
| Docosanol | 3.0 | |
| Stearic acid | 3.0 | |
| Batilol | 1.0 | |
| Glycerol stearate | 1.0 | |
| Polyoxyethylene sorbitol | 2.0 | |
| tetra-oleate | | |
| Hydrogenated soy lecithin | 1.0 | |
| Ceramide | 0.1 | |
| Retinol palmitate | 0.1 | |
| Preservatives | Appropriate amounts | |
| Extracts of Brahmi | 1.0 | |
| The testosterone generating and | 1.0 | |
| metabolizing enhancer | | |
| 1,3-butylene glycol | 5.0 | |
| Purified water | For making the product | |
| | up to 100 wt % | |
| | 100.0 | wt % |

Formula 13: for a cream

| Squalane | 4.0 | wt % |
|---|---|---|
| Vaseline | 2.5 | |
| Batilol | 2.0 | |
| Glycerin | 2.0 | |
| Oil-type glycerol stearate | 1.0 | |
| Stearic acid | 1.0 | |
| L-arginine | 1.0 | |
| The testosterone generating and | 0.5 | |
| metabolizing enhancer | | |
| Potassium hydroxide | 0.1 | |
| Aroma chemicals | Minute amounts | |
| Purified water | For making the product | |
| | up to 100 wt % | |
| | 100.0 | wt % |

Formula 14: for a cleansing product (in liquid form)

| Propylene glycol | 50.0 | wt % |
|---|---|---|
| Ethanol | 20.0 | |
| Sodium sulfate | 5.0 | |
| The testosterone generating and | 0.5 | |
| metabolizing enhancer | | |
| Lanolin | 0.5 | |
| Shea butter | 0.5 | |
| Colorants | 1.5 | |
| Aroma chemicals | 22.0 | |
| | 100.0 | wt % |

Formula 15: for a cat food

| Corn powder | 34.0 | wt % |
|---|---|---|
| Wheat powder | 35.0 | |
| Meat powder | 15.0 | |
| Tallow | 8.9 | |
| Salt | 1.0 | |
| Extracts of the Bonito fish | 4.0 | |
| The testosterone generating and | 1.0 | |
| metabolizing enhancer | | |
| Taurine | 0.1 | |
| Vitamins | 0.5 | |
| Minerals | 0.5 | |
| | 100.0 | wt % |

Formula 16: for a dog food

| Corn powder | 30.0 | wt % |
|---|---|---|
| Meat (chicken) | 15.0 | |
| Non-fat soybean | 10.0 | |
| Wheat powder | 25.0 | |
| Wheat bran | 5.0 | |
| The testosterone generating and | 5.0 | |
| metabolizing enhancer | | |
| Animal fats | 8.9 | |
| Oligosaccharides | 0.1 | |
| Vitamins | 0.5 | |
| Minerals | 0.5 | |
| | 100.0 | wt % |

### INDUSTRIAL APPLICATIONS

As described previously, a testosterone generating enhancer is proposed in the invention, which enhances the gene expression of enzymes related to testosterone synthesis, and increases testosterone levels in the seminal vesicle and the bloodstream. In addition, a testosterone metabolizing enhancer is also proposed in the invention, which enhances the gene expression of 5α-reductase (which metabolizes testosterone into dihydrotestosterone), such that the metabolism of testosterone into dihydrotestosterone is promoted subsequently.

1. A method for enhancing generation of testosterone in a subject comprising administering a phenylethanoid glycoside as a potent component to the subject.

2. The method of claim 1, wherein said phenylethanoid glycoside comprises at least one of echinacoside and acteoside.

3. The of claim 2, wherein said phenylethanoid glycoside comprises both echinacoside and acteoside.

4. The method of claim 1 which comprises administering an extract from a plant of Cistanche family containing said phenylethanoid glycoside to the subject.

5. (canceled)

6. The method of claim 4, wherein said phenylethanoid glycoside of the extract comprises at least one of echinacoside and acteoside.

7. The method of claim 6, wherein said phenylethanoid glycoside of the extract comprises both echinacoside and acteoside.

8. A method for enhancing metabolism of testosterone in a subject comprising administering a phenylethanoid glycoside as a potent component to the subject.

9. The method of claim 8, wherein said phenylethanoid glycoside comprises at least one of echinacoside and acteoside.

10. The method of claim 9, wherein said phenylethanoid glycoside comprises both echinacoside and acteoside.

11. The method of claim 8 which comprises administering an extract from a plant of Cistanche family containing the phenylethanoid glycoside to the subject.

12. (canceled)

13. The method of claim 11, wherein said phenylethanoid glycoside of the extract comprises at least one of echinacoside and acteoside.

14. The method of claim 13, wherein said phenylethanoid glycoside of the extract comprises both echinacoside and acteoside.

15. (canceled)

16. The method of claim 1, wherein the subject as a human, and the phenylethanoid glycoside is administered to human skin.

17. The method of claim 1, wherein the subject is a human, and the phenylethanoid glycoside is administered orally.

18. The method of claim 1, wherein the subject is a mammal and the phenylethanoid glycoside is administered orally.

19. The method of claim 8, wherein the subject is a human, and the phenylethanoid glycoside is administered to human skin.

20. The method of claim 8, wherein the subject is a human, and the phenylethanoid glycoside is administered orally.

21. The method of claim 8, wherein the subject is a mammal, and the phenylethanoid glycoside is administered orally.
